(19) United States
(12) Patent Application Publication
(10) Pub. No.: US 2023/0119135 A1
(43) Pub. Date: Apr. 20, 2023

(54) PHARMACEUTICAL COMPOSITION FOR USE IN THE TREATMENT OF DYSMENORRHEA AND/OR PREMENSTRUAL SYNDROME (PMS)

(71) Applicant: Umberto Cornelli, Milano (IT)

(72) Inventor: Umberto Cornelli, Milano (IT)

(21) Appl. No.: 17/904,955

(22) PCT Filed: Feb. 23, 2021
(86) PCT No.: PCT/EP2021/054370

§ 371 Date: Aug. 25, 2022

(30) Foreign Application Priority Data
Feb. 26, 2020 (IT) 102020000003964

(51) Int. Cl. A61K 31/593; A61K 31/01; A61K 31/352; A61K 31/122; A61K 33/10; A61K 31/19; A61P 15/00

(52) U.S. Cl. CPC A61K 31/593; A61K 31/01; A61K 31/352; A61K 31/122; A61K 33/10; A61K 31/19; A61P 15/00

(57) ABSTRACT

The present invention relates to a pharmaceutical composition comprising at least one Vitamin of the group D, lycopene, at least one flavonoid, at least one terpene and at least one calcium salt of a (C₁-C₃) carboxylic acid for use in the treatment of one or more menstrual disorders selected from the group consisting of Dysmenorrhea and Premenstrual syndrome (PMS).

### FIELD OF THE INVENTION

The present invention relates to a pharmaceutical composition and a pharmaceutical formulation for use in the treatment of Dysmenorrhea or Premenstrual syndrome (PMS) or both disorders at the same time.

### STATE OF THE ART

Dysmenorrhea is a disease affecting about 45% of menstruating women (approximately between 45 to 95% of females) during the time of menses (lacovides et al. “What we know about primary Dysmenorrhea today: a critical review”, Hum. Rep. Update 2015; 1-17), causing an extensive personal and public health problem, such as a high degree of absenteeism and severe economic loss.

Dysmenorrhea causes sharp crampy pain, or may be cramping and throbbing or dull constant ache that may radiate to the legs.

Usually, it starts few days before and/or at the onset of menses, often consisting of headache, nausea, constipation, diarrhea or urinary frequency, and low back pain. Usually these symptoms are peaking in 24 hours before menses and persisting for 2-3 days after onset of menses. In some cases, symptoms may occur during part of all of the menses. The disease can be primary (the more common) or secondary (due to pelvic abnormalities).

In the primary form, symptoms cannot be explained by structural gynaecological disorders, and pain is thought to result from uterine contraction and ischemia, probably mediated by prostaglandins and other inflammatory mediators produced by secretory endometrium, and possibly associated with prolonged uterine contractions and decreased blood flow to the myometrium. Contributing factors may include the passage of menstrual tissue through the cervix, or anatomical characteristics such as a mispositioned uterus or a narrow cervical ostium.

At present, possible suggested therapies available are adequate rest, sleep, regular exercise, diet, and in case drug support.

In terms of diet, a low-fat diet, reach in ω3 fatty acids, flaxseed, magnesium, Vitamin E, Vitamin B1 can be potentially effective.

If the pain persists, prostaglandin inhibitors such as NSAIDs (analgesic drugs) are the current most common pharmacological treatment. Such drugs can be taken 24-48 before and continued 1 or 2 days after menses begins.

Normally NSAIDs are effective in the pain relief, however around 15% of women across the age range who suffer from Dysmenorrhea do not respond to, or are intolerant to PG-inhibitors (Rauh et al., 1985; Campbell and McGrath, 1999). Moreover it is widely known that the prolonged use of NSAIDs might cause stomach pain or ulcers or possible liver or kidney problems.

If the NSAIDs are not effective or cannot be used, suppression of ovulation with a low-dose estrogen/progestin oral contraception is advisable and is often used as second-line therapy. Other hormonal treatment, such as danazol, progestins (e.g. levonorgestrel, etonogestrel, depot methoxy-progesterone acetate) gonadotropins-releasing hormone agonists, may decrease symptoms of Dysmenorrhea.

The synthetic hormones in oral contraceptive suppress ovulation and reduce the thickness of the endometrial lining of the uterus, thereby reducing the volume of menstrual fluid, PG-synthesis and dysmenorrheic pain (Dawood, 1995).

However, it has been confirmed a long-suspected association between oral contraceptive use and the risk of venous thromboembolism (Manzoli et al. 2012) and their use in some women may therefore being contra-indicated.

In such cases, progesterone IUD (intra uterine device) is advisable, to reduce the severity of menstrual pain (Suhonen et al, 2004; Lindh and Milsom, 2013).

However, the use of said hormonal intra-uterine devices in nulliparous women is still relatively low and might be not advisable.

Other currently available therapeutic approaches include: hypnosis, acupuncture, chiropractic therapy and transcutaneous electrical nerve stimulations.

However, such therapeutical approaches are not considered to be enough effective, to be widely used in clinical practice (Khan et al., 2012), and RCTs showing efficacy of such approaches are limited (Proctor and Farquhar, 2006).

For intractable pain is therefore sometimes necessary presacral neurectomy or laparoscopic uterosacral nerve ablation surgery.

However, said therapies are invasive and painful for the patients and require time for the recovery after the surgery.

Regarding the Premenstrual syndrome (PMS), sometimes is difficult to determine the difference between Dysmenorrhea and Premenstrual syndrome (PMS) since both somatic and behavioural symptoms may be similar in both diseases. In the Premenstrual syndrome (PMS) usually some of the somatic and behavioural symptoms (particularly breast tenderness, headache, irritability and anger) are usually more severe than with Dysmenorrhea (Greene et al., “The premenstrual syndrome”, Br. Med. J., 1953, 9, 1007-1014) with more severe limitation of the daily activity.

Both menstrual disorders have an immediate negative impact on quality life, for up to a few days every month and women suffering from both disorders have a significantly reduced quality of life, poorer mood and poorer sleep quality during menstruation compared to women not affected.

At least 25% of menstruating women report moderate-to-severe premenstrual symptoms, and in approximately 5% of the cases those symptoms are reported as severe (Pearlstein et al. “Premenstrual dysphoric disorder: burden of illness and treatment update”, J Psychiatry Neuroscience, 2008, 33, 4, 291-301).

The suggested aetiology of PMS includes abnormal neurotransmitter responses to normal ovarian functions, hormonal imbalance, sodium retention, or nutritional deficiencies (O'Brien, “Helping women with premenstrual syndrome”, Br. Med. Jour., 1993, 307:1471-1475).

There is evidence of a relationship between the syndrome and changes in certain progesterone metabolites (pregnenolone and allopregnanolone) during the menstrual cycle, which may interfere with neurotransmitters, especially gamma-aminobutyric acid (GABA) and serotonin which are important regulator of stress, anxiety, and alertness. These aforementioned metabolites act as positive modulators of the GABAergic system in the brain and also in the gastrointestinal tract, and make less receptive the GABA receptor.

Depletion of serotonin levels is associated with anxiety and depressive symptoms, since the selective serotonin uptake inhibitors (SSRI) are found to be active in PMS, and it has been suggested that serotonin increase the sensitivity to progesterone. Due to the behavioural modifications, the pharmacological treatments have included antidepressants (selective serotonin inhibitors, SSRIs) and other psychotropic agents, diuretics, progesterone, GnRh agonists, hormonal therapy such as estrogen therapy, combined oral contraceptive, pyridoxine, ethinylestradiol and dosperidone, and synthetic androgen and gonadotropin inhibitors (De Monico et al., “Premenstrual syndrome”, Curr. Opin. Obstet. Gynecol., 1994, 6:499-502). However, other non-pharmaceutical approaches are available, including dietary changes, exercise, cognitive behavioural therapy, and complementary alternative medicine, which sometimes were shown to be effective (Kim et al., “Acupuncture for premenstrual syndrome: a systematic review and meta-analysis of randomized controlled trials”, BJOG 2011, 118:899-915).

The aforementioned known treatments for both menstrual disorders, namely Dysmenorrhea and Premenstrual syndrome (PMS), imply the administrations of hormonal treatments, analgesic drugs and anti-depressants.

In US2007/098819 a treatment and prevention of premenstrual syndrome (PMS) with a bolus dose supplement comprising a combination of calcium carbonate and vitamin D2 or D3 is described.

In GB2169202 a composition is described, which provides relief from menstrual stresses (experienced by females at puberty, during menstruation, and during pseudo menstrual cycles), The composition comprises 5000 I.U. of Vitamin D (fish oil source), 1 gram calcium carbonate, 400 mg magnesium hydroxide, 1 gram Vitamin C (calcium ascorbate), 1 gram pantothenic acid, 100 mg B6 (pyridoxine hydrochloride), 600 I.U. Vitamin E (d-alpha tocopherol), and binders.

These less severe treatments are judged not so efficacious, thus rendering the use of analgesic drugs the final solution.

Therefore it is still felt the need of providing alternative formulations, which can treat singularly or both disorders at the same time, without any side effects of the known therapies (such as stomach pain or ulcers and possible liver or kidney problems) or invasive surgeries, while avoiding hormonal therapies or psychotropic agents. Hence, the present invention aims at treating said menstrual disorders, specifically Dysmenorrhea or Premenstrual syndrome (PMS) or both disorders at the same time, without any side effects and with a good compliance for the patients.

### SUMMARY OF THE INVENTION

The inventors surprisingly found out that by combining at least one Vitamin of the group D with lycopene, at least one flavonoid, at least one terpene and at least one calcium salt of a (C₁-C₃) carboxylic acid in the form of pharmaceutical composition of the invention, it was possible to drastically reduce both somatic and behavioural symptoms in women affected by one or more menstrual disorders selected from the group consisting of Dysmenorrhea and Premenstrual syndrome (PMS), without any side effect of the known therapies (such as stomach pain or ulcers and possible liver or kidney problems) or invasive surgeries and without the need of hormonal therapies or psychotropic agents.

Hence, the present invention relates to a pharmaceutical composition comprising at least one Vitamin of the group D, lycopene, at least one flavonoid, at least one terpene and at least one calcium salt of a (C₁-C₃) carboxylic acid for use in the treatment of one or more menstrual disorders selected from the group consisting of Dysmenorrhea and Premenstrual syndrome (PMS).

A further object of the present invention is a pharmaceutical formulation comprising the pharmaceutical composition of the invention and at least one pharmaceutically acceptable vehicle, for use in the treatment of one or more menstrual disorders selected from the group consisting of Dysmenorrhea and Premenstrual syndrome (PMS).

Furthermore, in a preferred and advantageous aspect the present invention concerns a pharmaceutical formulation, preferably in the form of a capsule, comprising Vitamin D₃ in an amount of 4 μg, calcium carbonate in an amount of 262 mg, calcium lactate in an amount of 179 mg, lycopene in an amount of 1.6 mg, astaxanthin in an amount of 0.4 mg, citrus flavonoids in an amount of 53.3 mg and the suitable pharmaceutical excipients are in an amount of 70 mg.

Through the pharmaceutical composition of the invention and its formulation, preferably in the form of capsules, it is possible to treat one or more menstrual disorders selected from Dysmenorrhea and Premenstrual syndrome (PMS), more advantageously to treat both menstrual diseases at the same time, without any side effects and with a good compliance for the patients and without the need of any heavy drugs or surgical treatments.

In a still further advantageous aspect the invention relates to the pharmaceutical formulation of the invention for use in the treatment of a menstrual disease selected from Dysmenorrhea and Premenstrual syndrome (PMS) and both diseases, wherein the pharmaceutical formulation is administered with a daily dosage in the range from 500 mg to 1.5 g, preferably from 1 g to 1.2 g.

In a preferred aspect the pharmaceutical formulation for use corresponds to a daily dosage of two capsules of the invention.

### DETAILED DESCRIPTION OF THE INVENTION

The present invention relates to a pharmaceutical composition comprising at least one Vitamin of the group D, lycopene, at least one flavonoid, at least one terpene and at least one calcium salt of a (C₁-C₃) carboxylic acid, for use in the treatment of one or more menstrual disorders selected from the group consisting of Dysmenorrhea and Premenstrual syndrome (PMS).

The inventors hence surprisingly found out that by combining at least one specific Vitamin of the group D, preferably Vitamin D₃ (cholecalciferol) with lycopene, at least one flavonoid, at least one terpene and at least one calcium salt of a (C₁-C₃) carboxylic acid in the form of pharmaceutical formulation of the invention, it was possible to drastically reduce both somatic and behavioural symptoms in women affected by Dysmenorrhea or Premenstrual syndrome (PMS) or both disorders at the same time, without the need of administering hormonal treatments, analgesic drugs and anti-depressants, having well-known side effects such as stomach pain or ulcers and possible liver or kidney problems, and avoiding furthermore any invasive surgeries.

Without being bound to any theory, the inventors found out that both the diseases, Dysmenorrhea and Premenstrual syndrome (PMS), can be treated by counteracting their inflammatory status, which is a common phenomenon for both. Therefore surprisingly according to the invention patients having also both diseases could be treated with surprising results as demonstrated in the experimental part.

For the purposes of the present invention, the following terms have the meaning herein reported:
“Dysmenorrhea” is intended as the phenomenon of uterine pain around the time of menses (The Merck Manual 19th Ed 2011);
“Premenstrual syndrome (PMS)” is intended as the phenomenon characterized by irritability, anxiety, emotional lability, depression, edema, breast pain, and headaches occurring during 7 to 10 day before and usually ending a few hours after onset of menses (The Merck Manual 19th Ed 2011);
“vehicle” as used herein indicates a medium, diluent with which the association of therapeutic or active ingredients is administered, for example water or any other pharmaceutically acceptable vehicle;
“pharmaceutically acceptable” identifies a substance that can be used within the area of pharmaceutical formulations. A physiologically acceptable vehicle/excipient may be for instance a pharmaceutically acceptable vehicle.

The pharmaceutical composition for use of the invention comprises at least one Vitamin of the group D. The vitamins D are a group of fat-soluble secosteroids. In the group D according to the invention Vitamin D₃ (also known as cholecalciferol) and Vitamin D₂ (ergocalciferol) are preferred, more preferably the Vitamin D is Vitamin D₃ (cholecalciferol), having formula (3β,5Z,7E)-9,10-secocholesta-5,7,10(19)-trien-3-ol.

In a preferred embodiment of the pharmaceutical composition for use of the invention, said Vitamin D₃ is in an amount in the range from 2 to 10 μg, preferably is in an amount in the range from 3 to 6 μg, more preferably is in an amount of 4 μg. The pharmaceutical composition for use of the invention comprises lycopene, at least one flavonoid and at least one terpene. In a preferred embodiment of the pharmaceutical composition for use of the invention, lycopene is in an amount in the range from 1 mg to 4 mg, preferably it is in an amount in the range from 1 to 3 mg, more preferably it is in an amount of about 1.6 mg.

The pharmaceutical composition for use of the invention comprises at least one flavonoid. Preferably said flavonoid is a citrus flavonoid. In a preferred embodiment of the pharmaceutical composition for use of the invention, said citrus flavonoid is present in an amount in the range from 30 to 100 mg, preferably is in an amount in the range from 40 to 80 mg, more preferably it is in an amount of about 53.3 mg. The pharmaceutical composition for use of the invention comprises at least one terpene. Preferably said terpene is astaxanthin.

In a preferred embodiment of the composition for use of the invention, astaxanthin is present in an amount in the range from 0.1 to 0.8 mg, preferably in an amount in the range from 0.2 to 0.6 mg, more preferably it is in an amount of about 0.4 mg. The pharmaceutical composition for use of the invention comprises at least one calcium salt of a (C₁-C₃) carboxylic acid. Preferably the at least one calcium salt is selected from the group consisting of calcium carbonate, calcium lactate and a mixture thereof.

In a preferred embodiment of the pharmaceutical composition for use of the invention, the calcium carbonate is in an amount in the range from 100 to 600 mg, preferably in an amount in the range from 200 to 300 mg, more preferably in an amount of about 262 mg.

In another preferred embodiment of the pharmaceutical composition for use of the invention, the calcium lactate is in an amount in the range from 80 to 400 mg, preferably in an amount in the range from 100 to 300 mg, more preferably in an amount of about 179 mg.

In further preferred embodiment of the pharmaceutical composition for use of the invention, said pharmaceutical composition for use further comprises at least one pharmaceutically acceptable excipient selected from the group consisting of magnesium salt, talcum, and a mixture thereof.

In a further aspect, the invention concerns a pharmaceutical formulation for use comprising the pharmaceutical composition for use of the invention and at least one pharmaceutically acceptable vehicle, for use in the treatment of one or more menstrual disorders selected from the group consisting of Dysmenorrhea and Premenstrual syndrome (PMS).

Preferably, said pharmaceutical formulation for use of the invention is selected from the group consisting of powders, granules, a capsule, and a tablet. More preferably the formulation for use of the invention is a capsule.

The present invention further concerns a capsule comprising Vitamin D₃ in an amount of 4 μg, calcium carbonate in an amount of 262 mg, calcium lactate in an amount of 179 mg, lycopene in an amount of 1.6 mg, astaxanthin in an amount of 0.4 mg, citrus flavonoids in an amount of 53.3 mg and at least one pharmaceutical excipient in an amount of 70 mg.

Advantageously, the pharmaceutical formulation for use of the invention has a daily dosage of said formulation in the range from 500 mg to 1.5 g, preferably from 1 g to 1.2 g.

In a preferred aspect the pharmaceutical formulation for use corresponds to a daily dosage of two capsules of the invention.

In a further aspect the invention relates to a method for the treatment of one or more menstrual disorders selected from the group consisting of Dysmenorrhea and Premenstrual syndrome (PMS) consisting in administering the pharmaceutical formulation of the invention to a human subject in a daily dosage of two capsules, each capsule comprising Vitamin D₃ in an amount of 4 μg, calcium carbonate in an amount of 262 mg, calcium lactate in an amount of 179 mg, lycopene in an amount of 1.6 mg, astaxanthin in an amount of 0.4 mg, citrus flavonoids in an amount of 53.3 mg and at least one pharmaceutical excipient in an amount of 70 mg. Preferably the two capsules are taken together, more preferably immediately before sleeping and at least two hours after the evening meal.

The present invention will be further illustrated by the following experimental part.

### EXPERIMENTAL PART

### Example 1

The Following Ingredients were Provided to Prepare a Single Capsule:

Pharmaceutical Composition:

| | | |
|---|---|---|
| Vitamin D₃ (cholecalciferol) | 4 | μg |
| Calcium carbonate | 262 | mg |
| Calcium lactate | 179 | mg |
| Lycopene | 1.6 | mg |
| Astaxanthin | 0.4 | mg |
| Citrus flavonoids | 53.3 | mg |
| Excipients | 70 | mg |

Specifically, the excipients were magnesium stearate and talcum.

The total amount of the ingredients above listed, in the form of powders were firstly filtered in a sieve with a knitted filter n. 16, and afterwards the powders were mixed for 15 minutes.

After the mixing step, the powders were introduced in a capsule, thus obtaining a formulation (F) in the form of capsule.

After encapsulation, the capsules were weighted, specifically ten capsules were weighted every 30 minutes, in order to check the uniformity and reproducibility of the average capsule content.

Then the capsules were de-pulverized through a knitted filter n. 6, thus obtaining the final capsules containing the formulation (F) of the invention.

### Example 2

Efficacy Test of the Treatment of Dysmenorrhea with the Formulation (F) of the Invention of Example 1 in the Form of a Capsule

The activity and effectiveness of the formulation of the invention in treating Dysmenorrhea was demonstrated experimentally by testing the formulation (F) on female subjects presenting the specific requirements herein provided.

The trial was conducted under the supervision of the Gynaecology Dept. Hospital-Mexico City.

In order to be admitted to the study, the subjects had to show at least 5 somatic and 5 behavioural symptoms of Dysmenorrhea listed in Table 1.

TABLE 1

| Somatic symptoms | Behavioral symptoms |
|---|---|
| Colic pain | Irritability |
| Bloatedness | Emotional tension |
| Brest tenderness | Anxiety |
| Intestinal modification | Mood swing |
| Body weight increase | Depression |
| Hot flashes | Aggression |
| Acne | Anger |
| Libido reduction | Fatigue |
| Back pain | Confusion/loss of |
| Food craving | concentration |
| Dizziness | Lethargy |
| Headache | Sadness |
| Thirst | Indecision |
| Nausea | Paranoia |
| Clumsiness | Over sensitivity |
| Fluid retention | Loss of motivation |
| | Crying easily |

Subjects were instructed to fill up a daily questionnaire relative to the symptoms, by mean of a semi-quantitative scale scoring from 0 to 4 to be completed during 3 subsequent menstrual cycles. Each symptom was scored from 0 to 4 as follows: Score 0=non present; 1=Mild (noticeable but not troublesome); 2=Moderate (interferes with normal activities); 3=Severe (unable to perform normal activities); 4=intolerable.

Cases with Dysmenorrhea appearing late in the menstrual phase were not admitted to the study. Subjects suffering from any cancer, chronic diseases such as Alzheimer's disease, depression, anorexia, paranoia, Chron's disease, bowel irritable syndrome, allergy or intolerance were not admitted to the study.

Other diseases such as hypertension, dyslipidemia were not within the exclusion criteria, provided that the therapy in place was effective, safe, and established by at least 3 months. Women under oral contraceptive treatment were not excluded in case the treatment was well tolerated and identical for at least 6 months.

The data were recorded during three subsequent menstrual cycles, and only those women reporting similar symptoms in the first two evaluations were admitted. The criteria used for admission were such that only 1 point of difference between the first two score evaluations (baseline and placebo) for any of the symptoms was accepted. In case of a scoring difference of >1 the subject was not admitted to the trial.

Twelve women of an age between 20 and 35 years were admitted because they were presenting the requirements above described.

The women general characteristics are summarized in Table 2.

TABLE 2

| | Mean | Standard |
|---|---|---|
| Variable | value | Deviation |
| Age [years] | 32 | 7.8 |
| Age of menarche [years] | 13 | 1.3 |
| Duration of menstruation [days] | 5 | 1.4 |

| General Characteristics | Frequency |
|---|---|
| School University degree/total | 4/12 |
| School Bachelor/total | 7/12 |
| Activity: professional/total | 11/12 |
| Activity: house wife/total | 0/12 |
| Activity: still medical student/total | 1/12 |
| Smoking/total | 0/12 |
| Concomitant therapy: oral contraceptive | 8/12 |
| Concomitant therapy: other | 0/12 |
| Previous therapy for Dysmenorrhea | 12/12 |

Three sets of measurements for the listed variables were made:
the first consisted of the baseline evaluation, and was done in the day of the symptom appearance, by the investigators together with the participants;
the second evaluation was done after the treatment with placebo, directly by the patients only; and
the third evaluation was done by the patient only during the treatment with the formulation (F) of the invention.

In the month immediately after the baseline evaluation, the treatment consisted of placebo in the quantity of two capsules per day, to be taken together, immediately before sleeping and at least two hours after the evening meal. The treatment was continued for 3 days before the expected menses.

The second treatment was done immediately following the next month consisting of 3 days, where the formulation (F) of the invention according to Example 1 was given in the amount of two capsules per day, to be taken together immediately before sleeping. The patients were instructed to take the formulation (F) of the invention according to Example 1 at least two hours after the evening meal.

Two boxes containing ten capsules (placebo or F) were given to each participant. The capsules of both treatments (placebo and F) were identical for colour and weight.

The main variable was the sum of the score of both somatic and behavioural symptoms after the two treatments (placebo and F), while all the other variables (see Table 1) were considered as ancillary variables. The average values (Mean) were calculated for each variable following the placebo and the treatment with formulation (F) of the invention.

The differences between the values after the two treatments (placebo and F) were tested using the test U (Mann-Whitney) (t-test), to assess the statistical significance to the data. These calculations were performed via the JMP15 Pro of SAS Institute. The data obtained are summarized in Table 3.

TABLE 3

| Period | After placebo | | After (F) | |
|---|---|---|---|---|
| Measures | Mean | SD | Mean | SD |
| Somatic symptoms | | | | |
| Colic type pain | 3.3 | 0.75 | [symbol] | 0.49 |
| Bloatedness | 1.5 | 0.67 | [symbol] | 0.72 |
| Breast tenderness | 3.1 | 1.00 | [symbol] | 0.60 |
| Intestinal | 1.5 | 1.09 | [symbol] | 0.65 |
| modifications ^{a} | | | | |
| Body weight | 0.6 | 0.67 | 0 | 0 |
| increase | | | | |
| Hot flashes | 2.2 | 1.03 | [symbol] | 0.45 |
| Acne ^{b} | 1.4 | 1.24 | [symbol] | 0.49 |
| Libido reduction | 2.1 | 1.83 | [symbol] | 0.67 |
| Back pain | 2.6 | 0.79 | [symbol] | 0.65 |
| Food craving | 0.4 | 0.67 | 0.4 | 0.51 |
| Dizziness | 1.8 | 0.72 | [symbol] | 0 |
| Headache | 3.5 | 0.80 | [symbol] | 0.62 |
| Thirst | 1.3 | 0.65 | [symbol] | 0.51 |
| Nausea | 1.3 | 0.75 | [symbol] | 0.39 |
| Clumsiness | 1.0 | 0.85 | [symbol] | 0.29 |
| Fluid retention | 1.0 | 0.74 | [symbol] | 0.62 |
| Total score somatic | 28.5 | 5.93 | 7.5^{c} | 2.58 |
| Behavioral | | | | |
| symptoms | | | | |
| Irritability | 3.2 | 0.72 | [symbol] | 0.71 |
| Emotional tension | 2.3 | 0.78 | [symbol] | 0.49 |
| Anxiety | 2.2 | 0.72 | [symbol] | 0.39 |
| Mood swing | 2.6 | 0.79 | [symbol] | 0.39 |
| Depression | 2.0 | 1.04 | [symbol] | 0.39 |
| Aggressiveness | 1.7 | 0.98 | [symbol] | 0.45 |
| Anger | 2.9 | 0.67 | [symbol] | 0.51 |
| Fatigue | 1.8 | 0.83 | [symbol] | 0.39 |
| Loss of | 2.0 | 1.04 | [symbol] | 0.29 |
| concentration | | | | |
| Lethargy | 1.4 | 0.79 | [symbol] | 0.29 |
| Sadness | 2.5 | 0.67 | [symbol] | 0.39 |
| Indecision | 2.0 | 0.60 | [symbol] | 0.29 |
| Paranoia | 1.4 | 0.90 | [symbol] | 0.29 |
| Over sensitivity | 2.1 | 0.67 | [symbol] | 0.45 |
| Loss of motivation | 2.1 | 1.08 | [symbol] | 0.29 |
| Crying easily | 1.6 | 0.90 | [symbol] | 0.29 |
| Total score | 33.8 | 9.87 | 7.2^{c} | 3.30 |
| behavioral | | | | |

^{a} = both constipation and diarrhea;
^{b} = exacerbation of the symptom in case of the presence;
^{c}= t test Placebo Vs formulation (F) of the invention, p < 0.05; [symbol]
= U Mann-Whitney test placebo (t-test) Vs formulation (F) of the invention, p < 0.05.

The mean values for each variable symptom after the placebo administration have been compared with respect to the mean values for each variable symptom after the administration of the formulation (F) of the invention.

By subtracting the mean value after the administration of the formulation (F) of the invention and the mean value after the administration of the placebo and by transforming the value into percentage, the inhibition power of the formulation (F) of the invention has been obtained, and the values are summarized in Table 4.

TABLE 4

| Period | |
|---|---|
| Measures | Inhibition power (%) |
| Somatic symptoms | |
| Colic type pain | 79 |
| Bloatedness | 72 |
| Breast tenderness | 68 |
| Intestinal | 56 |
| modifications ^{a} | |
| Body weight increase | 100 |
| Hot flashes | 65 |
| Acne ^{b} | 76 |
| Libido reduction | 56 |
| Back pain | 74 |
| Food craving | 0 |
| Dizziness | 100 |
| Headache | 79 |
| Thirst | 69 |
| Nausea | 87 |
| Clumsiness | 92 |
| Fluid retention | 75 |
| Total score somatic | 74 |
| Behavioral | |
| symptoms | |
| Irritability | 71 |
| Emotional tension | 86 |
| Anxiety | 62 |
| Mood swing | 68 |
| Depression | 92 |
| Aggressiveness | 85 |
| Anger | 63 |
| Fatigue | 91 |
| Loss of concentration | 71 |
| Lethargy | 94 |
| Sadness | 67 |
| Indecision | 96 |
| Paranoia | 94 |
| Over sensitivity | 64 |
| Loss of motivation | 96 |
| Crying easily | 95 |
| Total score | 79 |
| behavioral | |
| symptoms | |

From the above Tables is possible to observe that the total scoring following the treatment with the placebo and after the treatment with the formulation (F) of the invention were significantly different (t-test p<0.001) in favor of the formulation (F) of the invention, accounting for an average reduction of symptoms of 79%. Furthermore, before the treatments with the formulation (F) of the invention, the total scoring was higher for behavioral compared to somatic symptoms (respectively 33.8±9.87 and 28.5±5.93) but the differences were not statistically significant (t-test p>0.05).

All the variables were affected following the treatment with the formulation (F) of the invention, with different efficiency in reducing symptoms from 56% (libido reduction) up to 100% (dizziness).

Apart from the dizziness (100% inhibition), and the nausea (87% inhibition), the most affected symptoms were behavioral, specifically indecision, loss of motivation, crying easily, lethargy, paranoia, depression, fatigue, emotional tension, and aggressiveness. All these symptoms were reduced between 96% and 85% after treatment with the formulation (F) of the invention.

In conclusion, the present results indicate that surprisingly the formulation (F) of the invention is particularly effective both in reducing somatic and behavioral symptoms characteristic of Dysmenorrhea.

### Example 3

Efficacy Test of the Treatment of Premenstrual Syndrome (PMS) with the Formulation of the Invention (F) of Example 1

The activity and effectiveness of the formulation (F) of the invention in treating Premenstrual syndrome (PMS) was demonstrated experimentally by testing the pharmaceutical formulation (F) on subjects presenting the specific requirements herein provided.

The trial was conducted under the supervision of the Gynaecology Dept. Hospital-Mexico City.

In order to be admitted to the study, the subjects had to show at least 5 somatic and 5 behavioural symptoms of Premenstrual syndrome (PMS) listed in Table 5.

TABLE 5

| Somatic symptoms | Behavioral symptoms |
|---|---|
| Colic pain | Irritability |
| Bloatedness | Emotional tension |
| Brest tenderness | Anxiety |
| Intestinal modification | Mood swing |
| Body weight increase | Depression |
| Hot flashes | Aggression |
| Acne | Anger |
| Libido reduction | Fatigue |
| Back pain | Confusion/loss of |
| Food craving | concentration |
| Dizziness | Lethargy |
| Headache | Sadness |
| Thirst | Indecision |
| Nausea | Paranoia |
| Clumsiness | Over sensitivity |
| Fluid retention | Loss of motivation |
| | Crying easily |

Subjects were instructed to fill up a daily questionnaire relative to the symptoms, by mean of a semi-quantitative scale scoring from 0 to 4 to be completed during 3 subsequent menstrual cycles. Each symptom was scored from 0 to 4 as follows: Score 0=non present; 1=Mild (noticeable but not troublesome); 2=Moderate (interferes with normal activities); 3=Severe (unable to perform normal activities); 4=intolerable.

Subjects suffering from any cancer, chronic diseases such as Alzheimer's disease, depression, anorexia, paranoia, Chron's disease, bowel irritable syndrome, allergy or intolerance were not admitted to the trial. Other diseases such as hypertension, dyslipidemia were not within the exclusion criteria provided that the therapy in place was, effective, safe, and established by at least 3 months.

Women under oral contraceptive treatment were not excluded only in case that the treatment was safe and identical for at least 6 months.

The data were recorded during three subsequent menstrual cycles, and only those women reporting similar symptoms in the first two evaluations were admitted. The criteria used for admission were such that only 1 point of difference between the first two score evaluations (baseline and placebo) for any of the symptoms was accepted. In case of a scoring difference of >1 the subject was not admitted to the trial.

Twenty-two women of an age between 14 and 45 years were admitted because they were presenting the requirements above described.

The women general characteristics are summarized in Table 6.

TABLE 6

| | | Standard |
|---|---|---|
| Variable | Mean | Deviation |
| Age [years] | 34 | 11.6 |
| Age of menarche [years] | 13 | 1.3 |
| Duration of menstruation [days] | 5 | 1.4 |

| | Frequency |
|---|---|
| School University degree/total | 6/22 |
| School Bachelor/total | 12/22 |
| Activity: professional/total | 18/12 |
| Activity: house wife/total | 0/22 |
| Students | 4/22 |
| Smoking/total | 0/22 |
| Concomitant therapy: oral contraceptive | 16/22 |
| Concomitant therapy: other | 2/22 |
| Previous therapy for Premenstrual syndrome | 18/22 |
| (PMS) | |

Three sets of measurements for the listed variables were made:
the first consisted of the baseline evaluation, and was done in the day of the symptom appearance, by the investigators together with the participants;
the second evaluation was done after the treatment with placebo, directly by the patients only; and
the third evaluation was done by the patient only during the treatment with the formulation (F) of the invention.

In the month immediately after the baseline evaluation, the treatment consisted of placebo in the quantity of two capsules per day, to be taken together, immediately before sleeping and at least two hours after the evening meal. The treatment was continued for 3 days before the expected menses.

The second treatment was done immediately following next month consisting of 3 days, where the formulation (F) of the invention according to Example 1 was given in the amount of two capsules per day, to be taken together immediately before sleeping. The patients were instructed to take the formulation (F) of the invention according to Example 1 at least two hours after the evening meal.

Two boxes containing 10 cps (placebo or F) were given to each participant. The capsules of both treatments (placebo and F) were identical for color and weight. The main variable was the sum of the score of both somatic and behavioural symptoms after the two treatments (placebo and F), while all the other variables (see Table 5) were considered as ancillary variables. The average values (Mean) were calculated for each variable following the placebo and the treatment with formulation (F) of the invention.

The differences between the values after the two treatments (placebo and F) were tested using the test U (Mann-Whitney) (t-test), to assess the statistical significance to the data. These calculations were performed via the JMP15 Pro of SAS Institute. The data obtained are summarized in Table 7.

TABLE 7

| Period | After placebo | | After (F) | |
|---|---|---|---|---|
| Measures | Mean | SD | Mean | SD |
| Somatic variables | | | | |
| Colic type pain | 2.9 | 0.75 | [symbol] | 0.50 |
| Bloatedness | 1.7 | 0.77 | [symbol] | 0.50 |
| Breast tenderness | 2.4 | 0.79 | [symbol] | 0.49 |
| Intestinal | 1.9 | 0.87 | [symbol] | 0.47 |
| modifications a | | | | |
| Bodyweight | 0.5 | 0.67 | [symbol] | 0.27 |
| increase | | | | |
| Hot flashes | 2.2 | 0.80 | [symbol] | 0.49 |
| Acne ^{b} | 1.3 | 1.04 | [symbol] | 0.71 |
| Libido reduction | 2.0 | 1.50 | [symbol] | 0.60 |
| Back pain | 2.2 | 0.50 | [symbol] | 0.51 |
| Food craving | 1.0 | 0.82 | [symbol] | 0.57 |
| Dizziness | 1.6 | 0.73 | [symbol] | 0.33 |
| Headache | 2.6 | 0.50 | [symbol] | 0.58 |
| Thirst | 1.4 | 0.67 | [symbol] | 0.51 |
| Nausea | 1.5 | 0.74 | [symbol] | 0.20 |
| Clumsiness | 1.0 | 0.76 | [symbol] | 0.27 |
| Fluid retention | 1.0 | 0.69 | [symbol] | 0.53 |
| Total score somatic | 27.2 | 5.27 | 6.9 ^{c} | 2.71 |
| Behavioral variables | | | | |
| Irritability | 2.5 | 0.74 | [symbol] | 0.43 |
| Emotional tension | 2.3 | 0.63 | [symbol] | 0.56 |
| Anxiety | 2.1 | 0.64 | [symbol] | 0.40 |
| Mood swing | 2.0 | 0.79 | [symbol] | 0.40 |
| Depression | 2.0 | 0.98 | [symbol] | 0.46 |
| Aggressiveness | 1.4 | 1.18 | [symbol] | 0.57 |
| Anger | 2.2 | 0.75 | [symbol] | 0.48 |
| Fatigue | 2.4 | 0.73 | [symbol] | 0.55 |
| Lossof | 1.9 | 0.94 | [symbol] | 0.50 |
| concentration | | | | |
| Lethargy | 1.4 | 0.91 | [symbol] | 0.37 |
| Sadness | 2.3 | 0.89 | [symbol] | 0.20 |
| Indecision | 1.9 | 0.75 | [symbol] | 0.27 |
| Paranoia | 1.8 | 1.11 | [symbol] | 0.43 |
| Over sensitivity | 2.0 | 0.76 | [symbol] | 0.37 |
| Loss of motivation | 2.2 | 0.91 | [symbol] | 0.40 |
| Crying easily | 1.7 | 0.99 | [symbol] | 0.33 |
| Totalscore | 32.7 | 10.91 | 8.3 ^{c} | |
| behavioral | | | | |

^{a} = both constipation and diarrhea; ^{b} = exacerbation of the symptom in case of the presence; ^{c} = t test Placebo Vs formulation (F) of the invention, p < 0.05; [symbol] = U Mann-Whitney test (t-test) placebo Vs formulation (F) of the invention, p < 0.05.

The mean values for each variable symptom after the placebo administration have been compared with respect to the mean values for each variable symptom after the formulation (F) of the invention administration.

By subtracting the mean value after the administration of the formulation (F) of the invention and the mean value after the administration of the placebo and by transforming the value into percentage, the inhibition power of the formulation (F) of the invention has been obtained, and the values are summarized in Table 8.

TABLE 8

| Period | |
|---|---|
| Measures | Inhibition power (%) |
| Somatic | |
| symptoms | |
| Colic type pain | 83 |
| Bloatedness | 76 |
| Breast tenderness | 66 |
| Intestinal | 65 |
| modifications a | |
| Body weight | 86 |
| increase | |
| Hot flashes | 63 |
| Acne ^{b} | 62 |
| Libido reduction | 64 |
| Back pain | 77 |
| Food craving | 52 |
| Dizziness | 93 |
| Headache | 82 |
| Thirst | 65 |
| Nausea | 97 |
| Clumsiness | 92 |
| Fluid retention | 73 |
| Total score somatic | 75 |
| Behavioral | |
| symptoms | |
| Irritability | 69 |
| Emotional tension | 71 |
| Anxiety | 63 |
| Mood swing | 70 |
| Depression | 92 |
| Aggressiveness | 73 |
| Anger | 52 |
| Fatigue | 71 |
| Loss of | 77 |
| concentration | |
| Lethargy | 89 |
| Sadness | 55 |
| Indecision | 96 |
| Paranoia | 93 |
| Over sensitivity | 58 |
| Loss of motivation | 91 |
| Crying easily | 93 |
| Total score | 75 |
| behavioral | |
| symptoms | |

From the above Tables it can derive that the total scoring following the treatment with the placebo and after the treatment with the formulation (F) of the invention were significantly different (t-test p<0.001) in favor of the formulation (F) of the invention, accounting for an average reduction of symptoms of 75%.

Furthermore, before the treatments with the formulation (F) of the invention, the total scoring was higher for behavioral compared to somatic symptoms (respectively 32.7±10.91 and 27.2±5.27) and the differences were statistically significant (t-test p<0.05).

All the variables were affected following the treatment with the formulation (F) of the invention, with different efficiency in reducing symptoms from 55% (sadness reduction) up 97% (nausea).

The most affected symptoms were dizziness, nausea, clumsiness, depression, indecision, paranoia, loss of motivation, and crying easily, for which the reduction was >90%.

In conclusion, the present results indicate that surprisingly the formulation (F) of the invention is particularly effective both in reducing somatic and behavioral symptoms characteristic of Premenstrual syndrome (PMS).

### Example 4

Compliance Test of the Formulation of the Invention (F) for Dysmenorrhea and Premenstrual Syndrome (PMS)

The compliance of the formulation (F) of the invention was tested for the same subjects selected for Examples 2 and 3.

In order to be admitted to the study, the subjects had to show at least 5 somatic and 5 behavioural symptoms of Dysmenorrhea and Premenstrual syndrome (PMS) listed in Table 1 of Example 2 and Table 5 of Example 3.

The compliance test consisted in giving the subjects two boxes containing each 10 capsules of placebo and the formulation (F) of the invention according to Examples 1 and 2. The capsules of both treatments (placebo and F) were identical for colour and weight, therefore the subjects were not able to distinguish them from the appearance.

After the treatment, the residual capsules for both placebo and (F) were counted. The patients resulted to be not able to distinguish between the two types of capsules and finished the therapy without reporting any side effect or adverse reaction. Therefore, the formulation (F) of the invention showed to have an excellent compliance for the subject tested, being not recognizable from the placebo and not inducing any undesired effect.

### Example 5

Comparison Between the Formulation of the Invention (F) and Prior Art Formulation Comprising Calcium and Vitamin D

Twenty females affect by PMS were admitted to the investigation. They were treated with the Formulation F reported in Example 1 in comparison to a formulation containing only calcium (262 mg calcium carbonate+179 mg of calcium lactate), Vitamin D (cholecalciferol 4μ) and the same excipients (partial formula). The experience followed a double-blind controlled design. The subjects were all active in the medical field (doctors and nurses) and their age is reported in Table 1.

All the subjects were affected by PMS since at least one year, and were under treatment with oral contraceptive and the common painkillers. The experience was conducted under the supervision of the Gynecology dept, Hospital Mexico City.

Protocol Outline

The protocol was a double blind randomized design, and consisted of the evaluation of the daily discomfort of two different immediately following menses.

Admission Criteria

Women aging between 18 to 30 years, suffering from PMS from at least 1 year; PMS should have been characterized by at least 5 somatic and 5 behavioral symptoms (see example 2).

The subject were instructed for the evaluation of the daily discomfort. Only subjects reporting values of the daily discomfort ≥3 (see later) in the first day of symptom appearance were admitted.

Exclusion Criteria

Women with daily discomfort <3 in the first day of the symptom appearance. Women with a total score of daily discomfort <5 summing up the scores of the three days of the baseline investigation.

Any disease, or treatments with products different from oral contraceptive and pain killers.

The subjects were asked to substitute the usual painkillers with one of the two formulations under study.

The capsules and the boxes of the two formulations were identical. Each subject received 2 boxes containing 10 capsules (cps) to be taken as follows:

First day: 2 cps at moment of symptoms appearance, followed by other 2 cps at 6 hours of distance.

In case of need other 2 cps could have been taken at 4 hours of distance.

The second and the third day: 2 cps in the morning, and in case of need 2 cps after 6 hours and further 2 cps after 4 hours.

The subjects were asked to avoid possibly the use of any pain killers during the three-day experience.

In case of need, they should record the use. Every pain killer was accepted.

The daily discomfort was measured for three days using a VAS (Visual Analogue Scale) from 0 to 5 where:

0=no discomfort; 1=minimal discomfort compatible with all the daily activity; 2=moderate discomfort compatible with all the daily activity; 3=severe discomfort still compatible with the daily activity; 4=severe discomfort partially limiting the daily activity; 5=severe discomfort completely limiting the daily activity.

The baseline values of the discomfort used in the present investigation were those recorded in the month immediately before the treatment.

The 20 subjects were distributed random (via computer) to one of the two treatments.

Data Reporting

Subjects were instructed to report on a small record form the data concerning the daily discomfort, the pain killers use, and the number of capsules of the experimental formulas taken.

Statistical Analysis

The evaluation consisted in comparing the total scores values as sum of the three days of the treatments.

The daily discomfort total score was the main variable, while the use of pain killers was considered as an ancillary variable. Considering the data of previous experience in comparison to placebo, a number of 8 cases was sufficient to discriminate for α=0.05 and 1-β=0.8. The averages ±SD were calculated for all the variables. The t test was used to determine the scores difference between groups, while for the number of capsules the U Mann Whitney test was used. The compliance was measured on the base of the residual capsules.

Results

The compliance was excellent and no side effect were reported.

The age of the subjects are reported in Table 9.

TABLE 9
Age of the two groups of females suffering from PMS

| | | Comparison | |
|---|---|---|---|
| Formulation F | | Formulation | |
| | Age | | Age |
| N | years | N | years |
| 1 | 18 | 1 | 22 |
| 2 | 21 | 2 | 21 |
| 3 | 22 | 3 | 26 |
| 4 | 23 | 4 | 28 |
| 5 | 19 | 5 | 23 |
| 6 | 22 | 6 | 24 |
| 7 | 25 | 7 | 18 |
| 8 | 26 | 8 | 21 |
| 9 | 26 | 9 | 22 |
| 10 | 27 | 10 | 23 |
| M | 22.9 | M | 22.8 |
| SD | 2.50 | SD | 2.00 |

| | |
|---|---|
| T test | 0.940 p > 0.05 |

None of the subjects was smoking, and all the subjects were familiar with the use of pain killers to control the symptoms.

TABLE 10
Daily discomfort: baseline data before the
treatment with the complete and partial formula
at baseline: values of three days and total scores

| Formulation F | | | | | | Comparison formulationj | | | | | |
|---|---|---|---|---|---|---|---|---|---|---|---|
| | Days | | | Total | Pain | | Days | | | Total | Pain |
| N | 1 | 2 | 3 | score | killers | N | 1 | 2 | 3 | score | killers |
| 1 | 4 | 4 | 3 | 11 | 4 | 1 | 4 | 3 | 2 | 9 | 4 |
| 2 | 5 | 5 | 3 | 13 | 4 | 2 | 5 | 5 | 2 | 12 | 5 |
| 3 | 5 | 5 | 3 | 13 | 4 | 3 | 4 | 4 | 3 | 11 | 5 |
| 4 | 4 | 3 | 2 | 9 | 4 | 4 | 4 | 3 | 2 | 9 | 4 |
| 5 | 5 | 4 | 2 | 11 | 5 | 5 | 4 | 3 | 2 | 9 | 4 |
| 6 | 5 | 4 | 3 | 12 | 5 | 6 | 4 | 4 | 3 | 11 | 5 |
| 7 | 5 | 4 | 2 | 11 | 5 | 7 | 5 | 4 | 2 | 11 | 5 |
| 8 | 4 | 4 | 3 | 11 | 5 | 8 | 3 | 3 | 3 | 5 | 5 |
| 9 | 4 | 3 | 2 | 9 | 4 | 9 | 4 | 3 | 2 | 5 | 5 |
| 10 | 3 | 3 | 2 | 8 | 4 | 10 | 4 | 3 | 2 | 9 | 5 |
| M | | | | 10.8^{a} | 4.7 | | | | | 9.9 | 4.5^{b} |
| SD | | | | 1.28 | 0.48 | | | | | 1.08 | 0.50 |

^{a}= CF Vs PF; t test p > 0.05
^{b}= CF Vs PF; U Mann Whitney test p > 0.05;

The baseline data were those recorded in the month immediately before the treatment. The two groups were not statistically different (t test p>0.05). The use of pain killers was almost identical in the two groups (4 to 5 cps).

The data after the treatment with the two formulations are reported in Table 11.

TABLE 11
Daily discomfort: data after treatment with two different formulations:
Total number of capsules; values of three days and total scores

| Formulation F | | | | | | | Comparison Formulation | | | | | | |
|---|---|---|---|---|---|---|---|---|---|---|---|---|---|
| | Days | | | | | | | Days | | | | | |
| | 1 | 2 | 3 | | | | | 1 | 2 | 3 | | | |
| | Daily | | | | | | | Daily | | | | | |
| | dis- | | | Total | Pain | | | dis- | | | Total | Pain | |
| N | comfort | | | score | killers | cps | N | comfort | | | score | killers | cps |
| 1 | 2 | 0 | 0 | 2 | 0 | 10 | 1 | 3 | 3 | 2 | 8 | 3 | 18 |
| 2 | 3 | 0 | 0 | 3 | 0 | 10 | 2 | 3 | 2 | 2 | 8 | 3 | 14 |
| 3 | 2 | 0 | 0 | 2 | 0 | 8 | 3 | 4 | 3 | 2 | 9 | 3 | 18 |
| 4 | 2 | 0 | 0 | 2 | 0 | 10 | 4 | 4 | 3 | 2 | 9 | 4 | 14 |
| 5 | 2 | 0 | 0 | 2 | 0 | 8 | 5 | 5 | 2 | 2 | 9 | 3 | 18 |
| 6 | 0 | 1 | 0 | 1 | 0 | 10 | 6 | 5 | 3 | 2 | 10 | 4 | 14 |
| 7 | 0 | 1 | 0 | 1 | 0 | 10 | 7 | 4 | 3 | 2 | 9 | 4 | 18 |
| 8 | 0 | 0 | 0 | 0 | 0 | 8 | 8 | 3 | 3 | 3 | 9 | 3 | 14 |
| 9 | 2 | 1 | 0 | 0 | 0 | 10 | 9 | 5 | 2 | 2 | 9 | 4 | 18 |
| 10 | 1 | 0 | 0 | 0 | 0 | 8 | 10 | 3 | 2 | 2 | 7 | 4 | 14 |
| M | | | | 1.4^{a} | 0^{b} | 9.3^{b} | | | | | 8.6 | 3.5 | 16.0 |
| SD | | | | 0.92 | 0 | 1.03 | | | | | 0.76 | 0.53 | 2.11 |

^{a}= t test: CF Vs PF p < 0.01
^{b}= U Mann Whitney test: CF Vs PF < 0.01

The subjects under treatment with the Formulation F were taking as average less capsules than those under treatment with the comparison formulation, respectively 9±1.03 cps and 16±2.11 cps (p<0.01).

The Formulation F of the invention was found to be more effective (t test p<0.01) than the comparison formulation: the total daily discomfort scores following the treatment were respectively 1.4±0.92 and 8.6±0.76 (p<0.01).

The use of pain killers with the partial formula was significantly lower than the baseline values (3.5±0.53 Vs 4.7±0.48; U Mann Whitney p<0.05), while the Formulation F was free of any pain killer use.

A minimal activity was shown in the group treated with the comparison formulation (daily discomfort reduction from 9.9±1.08 to 8.6±0.76 (t test p<0.05).

The average daily amount of Calcium intake in the Comparison Formulation (summing up carbonate and lactate salts) was >1 g, while with the Formulation F it was about 0.6 g indicating that the addition of the other components (astaxanthin, citrus flavonoids, and lycopene) was surprisingly fundamental to determine the activity against the PMS symptoms.

### CONCLUSIONS

The Formulation F of the invention was found significantly more effective than the partial formula.

1. A method for treating one or more menstrual disorders selected from the group consisting of Dysmenorrhea and Premenstrual Syndrome (PMS) comprising the step of administering a pharmaceutical composition comprising at least one Vitamin of the group D, lycopene, at least one flavonoid, at least one terpene and at least one calcium salt of a (C1-C3) carboxylic acid.

2. The method according to claim 1, wherein the at least one Vitamin of the group D is Vitamin D3 (cholecalciferol), preferably in an amount in the range from 2 to 10 μg.

3. The method according to claim 1, wherein lycopene is in an amount in the range from 1 mg to 4 mg, preferably in an amount in the range from 1 to 3 mg, more preferably in an amount of about 1.6 mg.

4. The method according to claim 1, wherein the at least one flavonoid is a citrus flavonoid, preferably in an amount in the range from 30 to 100 mg, more preferably in an amount of about 53.3 mg.

5. The method according to claim 1, wherein the terpene is astaxanthin, preferably in an amount in the range from 0.1 to 0.8 mg, more preferably is in an amount of 0.4 mg.

6. The method according to claim 1, wherein the at least one calcium salt of a (C1-C3) carboxylic acid is selected from the group consisting of calcium carbonate, calcium lactate and a mixture thereof.

7. The method according to claim 6, wherein the calcium carbonate is in an amount in the range from 100 to 600 mg, preferably in an amount in the range from 200 to 300 mg, more preferably in an amount of about 262 mg.

8. The method according to claim 6, wherein the calcium lactate is in an amount in the range from 80 to 400 mg, preferably in an amount in the range from 100 to 300 mg, more preferably in an amount of about 179 mg.

9. A method for treating one or more menstrual disorders selected from the group consisting of Dysmenorrhea and Premenstrual Syndrome (PMS) comprising the step of administering a pharmaceutical formulation comprising the pharmaceutical composition according to claim 1 and at least one pharmaceutically acceptable vehicle.

10. The method according to claim 9, wherein said pharmaceutical formulation is selected from the group consisting of powders, granules, a capsule, and a tablet, preferably a capsule.

11. The method according to claim 9, wherein the daily dosage of the formulation is in the range from 500 mg to 1.5 g, preferably from 1 g to 1.2 g.
